(19) United States
(12) Patent Application Publication
(10) Pub. No.: US 2022/0184040 A1
(43) Pub. Date: Jun. 16, 2022

(54) METHOD OF TREATING MALIGNANT RHABDOID TUMOR OF THE OVARY AND SMALL CELL CANCER OF THE OVARY OF THE HYPERCALCEMIC TYPE

(71) Applicant: TRANSLATIONAL DRUG DEVELOPMENT, LLC, Scottsdale, AZ (US)

(72) Inventor: Stephen Gately, Scottsdale, AZ (US)

(21) Appl. No.: 17/598,074

(22) PCT Filed: Mar. 25, 2020
(86) PCT No.: PCT/US2020/024787

§ 371 Date: Sep. 24, 2021

Related U.S. Application Data
(60) Provisional application No. 62/826,270, filed on Mar. 29, 2019.
(60) Provisional application No. 62/824,275, filed on Mar. 26, 2019.

(51) Int. Cl. A61K 31/426; A61K 39/395; C07K 16/28; A61P 35/00

(52) U.S. Cl. CPC A61K 31/426; A61K 39/3955; A61K 2039/505; C07K 16/2827; A61P 35/00; C07K 16/2818

(57) ABSTRACT

The disclosure provides a method of treating a malignant rhabdoid tumor in a subject in need thereof including administering to the subject a therapeutically-effective amount of (S)-N-hydroxy-2-(2-(4-methoxyphenyl)butanamido)thiazole-5-carboxamide and pharmaceutically acceptable salts thereof. In certain embodiments of this method the malignant rhabdoid tumor is small cell cancer of the ovary of the hypercalcemic type (SCCOHT).

### CROSS-REFERENCE TO RELATED APPLICATIONS

This application claims priority to U.S. Provisional Application Nos. 62/824,275, filed Mar. 26, 2019, and 62/826,270, filed Mar. 29, 2019, the contents of each of which are incorporated herein by reference in their entireties.

### TECHNICAL FIELD

The disclosure is directed to the fields of small molecule therapies, cancer, and methods of treating rare cancer types.

### BACKGROUND

Small cell carcinoma of the ovary, hypercalcemic type (SCCOHT), is a rare, aggressive form of ovarian cancer diagnosed in young women. SCCOHT is generally fatal when spread beyond the ovary. SCCOHT represents less than 1% of all ovarian cancer diagnoses, with less than 300 cases reported in the literature to date. Estel et al., Arch Gynecol Obstet 284:1277-82 (2011) and Young et al., Am J Surg Pathol 18:1102-16 (1994). The mean age at diagnosis is 23 years and, unlike patients with the more common types of ovarian cancer, the majority of these women present with early-stage disease. Harrison et al., Gynecol Oncol 100:233-8 (2006). Nonetheless, most patients relapse and die within 2 years of diagnosis, regardless of stage, with a long-term survival rate of only 33%, even when disease is confined to the ovary at diagnosis. Seidman, Gynecol Oncol 59:283-7 (1995). There are no reliable adjuvant treatments that improve outcome, but multi-compound chemotherapy is thought to extend survival. Estel et al., Arch Gynecol Obstet 284:1277-82 (2011) and Pautier et al., Ann Oncol 18:1985-9 (2007).

The tissue of origin remains speculative, and SCCOHT is still categorized as a miscellaneous tumor by the World Health Organization. Most tumors are unilateral, and size greater than 10 cm may be prognostically favorable due to earlier onset of symptoms resulting in stage migration. Estel et al., Arch Gynecol Obstet 284:1277-82 (2011). Histologic classification can be challenging, but commonly expressed immunohistochemical markers such as CD10, WT1, and calretinin can be useful in conjunction with loss of detectable inhibin, 5100, and chromogranin expression to exclude histological mimics. McCluggage, Adv Anat Pathol 11:288-96 (2004).

Recent studies implicate the SWI/SNF (BAF) chromatin remodeling complex as a major tumor suppressor because frequent inactivating mutations in at least seven SWI/SNF subunits have been identified in a variety of cancers. The genes of the SWI/SNF complex were found to be associated with one of the first chromatin remodeling complexes to be identified, with many of its subunits conserved from yeast to humans. In mammalian cells, the SWI/SNF complex comprises of 11-15 protein subunits that include SNF5 (SMARCB1) and one of the two mutually exclusive ATPases, BRG1 (SMARCA4) or BRM (SMARCA2). Genetic alterations in subunits of the SWI/SNF chromatin-remodeling complex are a key mechanism in tumorigenesis of several cancers. This is exemplified by rhabdoid tumors, where frequent biallelic loss of the core SWI/SNF gene SMARCB1 is likely the primary driver of oncogenesis. Importantly, up to 20% of patients with rhabdoid tumors bear germline heterozygous mutations in SMARCB1 and inactivating germline mutations of SMARCA4 in patients lacking SMARCB1 mutations. At a somatic level, however, SMARCA4 is the SWI/SNF subunit most commonly mutated in cancer.

Although the mutational landscape of SCCOHT is unknown, the similarities between SCCOHT and rhabdoid tumors (both are highly aggressive pediatric tumors with primitive histologic features, diploid cytogenetics, and are sometimes familial) suggest they may have similar molecular genetics. There is a long-felt yet unmet need for effective treatments for certain cancers such as rhabdoid tumors and SCCOHT which may be caused by genetic alterations or loss of function of subunits of the SWI/SNF chromatin remodeling complex resulting in EZH2-dependent oncogenesis.

### SUMMARY

The disclosure provides effective treatments for INI1-negative and SMARCA4-negative tumors, such as malignant rhabdoid tumors (MRTs) and epithelioid sarcoma. INT1 and SMARCA4 are critical proteins of the SWItch/Sucrose NonFermentable (SWI/SNF) chromatin remodeling complex. In certain embodiments MRTs can be INI1-negative, INI1-deficient, SMARCA4-negative, SMARCA4 deficient, SMARCA2 negative, SMARCA2 deficient, or comprise a mutation on one or more other components of the SWI/SNF complex.

In some embodiments, the disclosure provides a method of treating a malignant rhabdoid tumor (MRT), a malignant rhabdoid tumor of the ovary (MRTO), and/or a small cell cancer of the ovary of the hypercalcemic type (SCCOHT) in a subject in need thereof, the method comprising: administering to the subject a therapeutically effective amount of N-hydroxy-2-(2-(4-methoxyphenyl)butanamido)thiazole-5-carboxamide or an enantiomer, pharmaceutically acceptable salt, solvate, or chemically protected form thereof.

In one embodiment, (S)-N-hydroxy-2-(2-(4-methoxyphenyl)butanamido)thiazole-5-carboxamide or a pharmaceutically acceptable salt thereof is administered to the subject. In another embodiment, the (S)-N-hydroxy-2-(2-(4-methoxyphenyl)butanamido)thiazole-5-carboxamide or pharmaceutically acceptable salt thereof is administered orally.

In some embodiments, the (S)-N-hydroxy-2-(2-(4-methoxyphenyl)butanamido)thiazole-5-carboxamide or pharmaceutically acceptable salt thereof is administered at a dose of between 1 mg/kg/day and 1600 mg/kg/day.

In other embodiments, the N-hydroxy-2-(2-(4-methoxyphenyl)butanamido)thiazole-5-carboxamide or enantiomer, pharmaceutically acceptable salt, solvate, or chemically protected form thereof is administered at a dose of about 100, 200, 400, 800, or 1600 mg per day.

In one embodiment, the SCCOHT is SMARCA4-negative. In another embodiment, the subject is SMARCA4-negative.

In certain embodiments, SMARCA4 expression is evaluated by a method comprising: (a) obtaining a biological sample from the subject; (b) contacting the biological sample or a portion thereof with an antibody that specifically binds SMARCA4; and (c) detecting an amount of the antibody that is bound to SMARCA4.

In other embodiments, SMARCA4 expression and/or function is evaluated by a method comprising: (a) obtaining a biological sample from the subject; (b) sequencing at least one DNA sequence encoding a SMARCA4 protein from the biological sample or a portion thereof and (c) determining if the at least one DNA sequence encoding the SMARCA4 protein contains a mutation affecting the expression and/or function of the SMARCA4 protein.

In yet other embodiments, the subject is less than 40 years of age, less than 30 years of age, less than 20 years of age, or between 20 and 30 years of age, inclusive of the endpoints. In one embodiment, the N-hydroxy-2-(2-(4-methoxyphenyl)butanamido)thiazole-5-carboxamide or enantiomer, pharmaceutically acceptable salt, solvate, or chemically protected form thereof prevents and/or inhibits proliferation of an SCCOHT cell.

In other embodiments, the disclosure provides a method of treating SCCOHT in a subject in need thereof, the method comprising administering to the subject in an oral tablet a therapeutically effective amount of N-hydroxy-2-(2-(4-methoxyphenyl)butanamido)thiazole-5-carboxamide or an enantiomer, pharmaceutically acceptable salt, solvate, or chemically protected form thereof. In one embodiment, the (S)-N-hydroxy-2-(2-(4-methoxyphenyl)butanamido)thiazole-5-carboxamide or a pharmaceutically acceptable salt thereof is administered to the subject.

In some embodiments, the present disclosure provides a method of treating MRT and/or MRTO in a subject in need thereof, the method comprising administering to the subject in an oral tablet a therapeutically-effective amount of N-hydroxy-2-(2-(4-methoxyphenyl)butanamido)thiazole-5-carboxamide or an enantiomer, pharmaceutically acceptable salt, solvate, or chemically protected form thereof.

In one embodiment, the MRT and/or MRTO is INI1-negative, INI1-deficient or epithelioid sarcoma.

In certain embodiments of the disclosure the MRT is malignant rhabdoid tumor of the ovary (MRTO), also referred to as small cell cancer of the ovary of the hypercalcemic type (SCCOHT). The disclosure provides a method of treating SCCOHT in a subject in need thereof comprising administering to the subject a therapeutically-effective amount of (S)-N-hydroxy-2-(2-(4-methoxyphenyl)butanamido)thiazole-5-carboxamide, its pharmaceutically acceptable salt, ester, derivative, analog, prodrug, or solvate thereof.

In certain embodiments of the disclosure the MRT is epithelioid sarcoma. The disclosure provides a method of treating epithelioid sarcoma in a subject in need thereof comprising administering to the subject a therapeutically-effective amount of (S)-N-hydroxy-2-(2-(4-methoxyphenyl)butanamido)thiazole-5-carboxamide, its pharmaceutically acceptable salt, ester, derivative, analog, prodrug, or solvate thereof.

(S)-N-hydroxy-2-(2-(4-methoxyphenyl)butanamido)thiazole-5-carboxamide may be administered orally. In certain embodiments, (S)-N-hydroxy-2-(2-(4-methoxyphenyl)butanamido)thiazole-5-carboxamide may be formulated as an oral tablet.

Methods of the disclosure for treating cancer in a subject in need thereof comprise administering a therapeutically-effective amount of (S)-N-hydroxy-2-(2-(4-methoxyphenyl)butanamido)thiazole-5-carboxamide to the subject. Determination of an effective amount of the disclosed compound is within the capability of those skilled in the art, especially in light of the detailed disclosure provided herein. The effective amount of a pharmaceutical composition used to affect a particular purpose as well as its toxicity, excretion, and overall tolerance may be determined in cell cultures or experimental animals by pharmaceutical and toxicological procedures either known now by those skilled in the art or by any similar method yet to be disclosed. One example is the determination of the IC₅₀ (half maximal inhibitory concentration) of the pharmaceutical composition in vitro in cell lines or target molecules. Another example is the determination of the LD₅₀ (lethal dose causing death in 50% of the tested animals) of the pharmaceutical composition in experimental animals. The exact techniques used in determining an effective amount will depend on factors such as the type and physical/chemical properties of the pharmaceutical composition, the property being tested, and whether the test is to be performed in vitro or in vivo. The determination of an effective amount of a pharmaceutical composition will be well known to one of skill in the art who will use data obtained from any tests in making that determination. Determination of an effective amount of disclosed compound for addition to a cancer cell also includes the determination of an effective therapeutic amount, including the formulation of an effective dose range for use in vivo, including in humans.

Methods of the disclosure for treating cancer including treating a malignant rhabdoid tumor (MRT). In preferred embodiments, methods of the disclosure are used to treat a subject having a malignant rhabdoid tumor of the ovary (MRTO). MRTO may also be referred to as small cell cancer of the ovary of the hypercalcemic type (SCCOHT). In certain embodiments, the MRTO or SCCOHT and/or the subject are characterized as SMARCA4-negative, SMARCA4 deficient, SMARCA2 negative, SMARCA2 deficient, or as having a mutation or deficiency in one or more other components of the SWI/SNF complex. In certain embodiments, the MRTO or SCCOHT and/or the subject are characterized as SMARCA4-negative. In certain embodiments, the MRTO or SCCOHT and/or the subject are characterized as SMARCA4-negative or SMARCA4-deficient; and SMARCA2-negative or SMARCA2-deficient. As used herein SMARCA4-negative and/or SMARCA4-deficient cells may contain a mutation in the SMARCA4 gene, corresponding SMARCA4 transcript (or cDNA copy thereof), or SMARCA4 protein, that prevents transcription of a SMARCA4 gene, translation of a SMARCA4 transcript, and/or decreases/inhibits an activity of a SMARCA4 protein. As used herein SMARCA4-negative cells may contain a mutation in the SMARCA4 gene, corresponding SMARCA4 transcript (or cDNA copy thereof), or SMARCA4 protein that prevents transcription of a SMARCA4 gene, translation of a SMARCA4 transcript, and/or decreases/inhibits an activity of a SMARCA4 protein.

Methods of the disclosure for treating cancer including treating a malignant rhabdoid tumor (MRT). In some preferred embodiments, methods of the disclosure are used to treat a subject having an epithelioid sarcoma. In certain embodiments, the epithelioid sarcoma is characterized as SMARCA4-negative, SMARCA4 deficient, SMARCA2 negative, SMARCA2 deficient, or as having a mutation or deficiency in one or more other components of the SWI/SNF complex. In certain embodiments, the epithelioid sarcoma and/or the subject are characterized as SMARCA4-negative. In certain embodiments, the epithelioid sarcoma and/or the subject are characterized as SMARCA4-negative or SMARCA4-deficient; and SMARCA2-negative or SMARCA2-deficient.

Methods of the disclosure may be used to treat a subject who is SMARCA4-negative or who has one or more cells that may be SMARCA4-negative. SMARCA4 expression and/or SMARCA4 function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds SMARCA4; and (c) detecting an amount of the antibody that is bound to SMARCA4. Alternatively, or in addition, SMARCA4 expression and/or SMARCA4 function may be evaluated by a method comprising: (a) obtaining a biological sample from the subject; (b) sequencing at least one DNA sequence encoding a SMARCA4 protein from the biological sample or a portion thereof; and (c) determining if the at least one DNA sequence encoding a SMARCA4 protein contains a mutation affecting the expression and/or function of the SMARCA4 protein. SMARCA4 expression or a function of SMARCA4 may be evaluated by detecting an amount of the antibody that is bound to SMARCA4 and by sequencing at least one DNA sequence encoding a SMARCA4 protein, optionally, using the same biological sample from the subject.

Subjects of the disclosure may be female. Subjects of the disclosure may be less than 40, 30, or 20 years of age. In certain embodiments, subjects of the disclosure may be between 20 and 30 years of age, inclusive of the endpoints.

In certain embodiments, the disclosure provides a combination comprising: (S)-N-hydroxy-2-(2-(4-methoxyphenyl)butanamido)thiazole-5-carboxamide or a pharmaceutically acceptable salt thereof; and an immune checkpoint molecule selected from the group consisting of an inhibitor of PD-1, an inhibitor of PD-L1, an inhibitor of LAG-3, an inhibitor of TIM-3, an inhibitor of CEACAM, and an inhibitor of CTLA-4.

In one aspect, the immune checkpoint molecule is an anti-PD-1 antibody molecule. In another aspect, the immune checkpoint molecule is an anti-PD-L1 antibody molecule. In another aspect, the immune checkpoint molecule is an anti-CTLA-4 antibody molecule. In another aspect, the immune checkpoint molecule is an anti-LAG-3 antibody molecule. In another aspect, the immune checkpoint molecule is an anti-TIM-3 antibody molecule. In another aspect, the immune checkpoint molecule is an anti-CEACAM antibody molecule. In another aspect, the immune checkpoint molecule is an antibody molecule against CEACAM-1, CEACAM-3, or CEACAM-5.

In one embodiment, administration of the (S)-N-hydroxy-2-(2-(4-methoxyphenyl)butanamido)thiazole-5-carboxamide or pharmaceutically acceptable salt thereof and the immune checkpoint molecule to a subject in need thereof provides a synergistic effect in the treatment of cancer.

### BRIEF DESCRIPTION OF THE DRAWINGS

FIG. 1 depicts growth inhibition of dual SMARCA2 and SMARCA4 deficient cell lines (i.e., A204, G401, G402, H522, and A427) with (S)-N-hydroxy-2-(2-(4-methoxyphenyl)butanamido)thiazole-5-carboxamide.

FIG. 2 depicts relative SMARCA2 gene expression in BIN-67 cells treated with (S)-N-hydroxy-2-(2-(4-methoxyphenyl)butanamido)thiazole-5-carboxamide (i.e., GB-3103).

FIG. 3 depicts SMARCA2 protein expression in BIN-67 cells treated with (S)-N-hydroxy-2-(2-(4-methoxyphenyl)butanamido)thiazole-5-carboxamide (i.e., GB-3103).

FIG. 4 depicts in vivo treatment of tumors in an SCCOHT xenograft model (BIN-67) with (S)-N-hydroxy-2-(2-(4-methoxyphenyl)butanamido)thiazole-5-carboxamide (i.e., GB-3103) for 60 days.

FIG. 5 depicts in vivo treatment of tumors in a malignant rhabdoid tumor xenograft model (G401) with (S)-N-hydroxy-2-(2-(4-methoxyphenyl)butanamido)thiazole-5-carboxamide (i.e., GB-3103) for 30 days.

FIG. 6 depicts the potent anti-proliferative activity of (S)-N-hydroxy-2-(2-(4-methoxyphenyl)butanamido)thiazole-5-carboxamide (i.e., GB-3103) against human SCCOHT lines BIN67, COV434, and SCCOHT-1.

FIG. 7 depicts the activity of (S)-N-hydroxy-2-(2-(4-methoxyphenyl)butanamido)thiazole-5-carboxamide (i.e., GB-3103) alone and in combination with anti-mPD-1 and anti-mPD-L1 antibodies in a syngeneic CT-26 mouse colon cancer model.

FIG. 8 depicts RNA-Seq analyses of BIN67 cells treated with (S)-N-hydroxy-2-(2-(4-methoxyphenyl)butanamido)thiazole-5-carboxamide (i.e., GB-3103).

FIG. 9 depicts measurements of increased expression of MHC Class I/II genes in BIN67 cells treated with (S)-N-hydroxy-2-(2-(4-methoxyphenyl)butanamido)thiazole-5-carboxamide (i.e., GB-3103).

### DETAILED DESCRIPTION

As used herein, the terms “(S)-N-hydroxy-2-(2-(4-methoxyphenyl)butanamido)thiazol-5-carboxamide,” “(2S)-N-[5-(hydroxycarbamoyl)thiazol-2-yl]-2-(4-methoxyphenyl)butanamide,” and “GB-3103” are synonymous and indicate the compound with the following chemical structure:

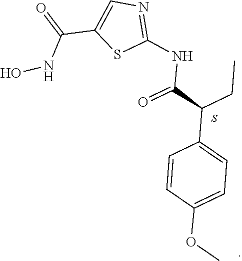

As used herein, the term “treating” may comprise preventing and/or inhibiting proliferation of a cancer cell, including, but not limited to a MRTO/SCCOHT cell.

INI1-negative and SMARCA4-negative tumors, such as malignant rhabdoid tumors (MRTs) and epithelioid sarcoma are serious and debilitating cancers. Approximately 1,400 patients each year in the major global markets develop these tumors, which have no established standard of care. INI1 and SMARCA4 are critical proteins of the SWI/SNF complex.

Exemplary cancers include malignant rhabdoid tumor of the ovary (MRTO), also referred to as small cell cancer of the ovary of the hypercalcemic type (SCCOHT).

A preferred method of treating MRTO (SCCOHT) in a subject in need thereof comprises administering to the subject a therapeutically-effective amount of (S)-N-hydroxy-2-(2-(4-methoxyphenyl)butanamido)thiazole-5-carboxamide.

(S)-N-hydroxy-2-(2-(4-methoxyphenyl)butanamido)thiazole-5-carboxamide of the disclosure is effective for treating cancers caused by a decreased abundance and/or function of a component of the SWI/SNF chromatin remodeling complex, including, for example, a decreased abundance and/or function of SMARCA4. Other components of the SWI/SNF complex that may become oncogenic markers or drivers are ARID1A, ARID2, ARID1B, SMARCB1, SMARCC1, SMARCA2, or SMARCD1. At a high-level view, the SWI/SNF chromatin remodeling complex uses ATP as a source of energy for opening the chromatin to provide access for gene transcription.

According to the methods of the disclosure, a “normal” cell may be used as a basis of comparison for one or more characteristics of a cancer cell, including expression and/or function of SMARCA4. As used herein, a “normal cell” is a cell that cannot be classified as part of a “cell proliferative disorder”. A normal cell lacks unregulated or abnormal growth, or both, that can lead to the development of an unwanted condition or disease. Preferably a normal cell contains a wild type sequence for the SMARCA4 gene, expresses a SMARCA4 transcript without mutations, and expresses a SMARCA4 protein without mutations that retains all functions at normal activity levels.

As used herein, “contacting a cell” refers to a condition in which a compound or other composition of matter is in direct contact with a cell or is close enough to induce a desired biological effect in a cell.

As used herein, “treating” or “treat” describes the management and care of a subject for the purpose of combating a disease, condition, or disorder and includes the administration of (S)-N-hydroxy-2-(2-(4-methoxyphenyl)butanamido)thiazole-5-carboxamide of the disclosure, or a pharmaceutically acceptable salt, prodrug, metabolite, polymorph or solvate thereof, to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute). Furthermore, severity describes a nuclear grade, which refers to the size and shape of the nucleus in tumor cells and the percentage of tumor cells that are dividing (see, National Cancer Institute).

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large. Ovarian cancers are considered silent killers because the cancer does not produce signs or symptoms severe enough to cause medical intervention until the tumors are either large or metastasized.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

An effective amount of (S)-N-hydroxy-2-(2-(4-methoxyphenyl)butanamido)thiazole-5-carboxamide of the disclosure, or a pharmaceutically acceptable salt, prodrug, metabolite, polymorph or solvate thereof, is not significantly cytotoxic to normal cells. For example, a therapeutically effective amount of (S)-N-hydroxy-2-(2-(4-methoxyphenyl)butanamido)thiazole-5-carboxamide of the disclosure is not significantly cytotoxic to normal cells if administration of the (S)-N-hydroxy-2-(2-(4-methoxyphenyl)butanamido)thiazole-5-carboxamide of the disclosure in a therapeutically effective amount does not induce cell death in greater than 10% of normal cells. A therapeutically effective amount of (S)-N-hydroxy-2-(2-(4-methoxyphenyl)butanamido)thiazole-5-carboxamide of the disclosure does not significantly affect the viability of normal cells if administration of the compound in a therapeutically effective amount does not induce cell death in greater than 10% of normal cells.

Contacting a cell with (S)-N-hydroxy-2-(2-(4-methoxyphenyl)butanamido)thiazole-5-carboxamide of the disclosure, or a pharmaceutically acceptable salt, prodrug, metabolite, polymorph or solvate thereof, can inhibit HDAC activity selectively in cancer cells. Administering to a subject in need thereof (S)-N-hydroxy-2-(2-(4-methoxyphenyl)butanamido)thiazole-5-carboxamide of the disclosure, or a pharmaceutically acceptable salt, prodrug, metabolite, polymorph or solvate thereof, can inhibit HDAC activity selectively in cancer cells.

### Malignant Rhabdoid Tumor

Malignant rhabdoid tumor (MRT) is a rare childhood tumor that occurs in soft tissues, most commonly starting in the kidneys, as well as the brain. A hallmark of certain malignant rhabdoid tumors is a loss of function of SMARCB1 (also known as INI1). INT1 is a critical component of the SWI/SNF regulatory complex, a chromatin remodeler that acts in opposition to EZH2. INI1-negative tumors have altered SWI/SNF function. This activity can be targeted by (S)-N-hydroxy-2-(2-(4-methoxyphenyl)butanamido)thiazole-5-carboxamide. INI1-negative tumors are generally aggressive and are poorly served by current treatments. For example, current treatment of MRT, a well-studied INI1-negative tumor, consists of surgery, chemotherapy and radiation therapy, which are associated with limited efficacy and significant treatment-related morbidity. The annual incidence of patients with INI1-negative tumors and synovial sarcoma in major markets, including the U.S., E.U. and Japan, is approximately 2,400. Loss of function of SMARCB1/INI1 also occurs in another rare and aggressive childhood tumor, atypical teratoid rhabdoid tumor (AT/RT) of the central nervous system.

### Malignant Rhabdoid Tumor of the Ovary MRTO (Small Cell Cancer of the Ovary of the Hypercalcemic Type (SCCOHT))

MRTO/SCCOHT is an extremely rare, aggressive cancer affecting children and young women (mean age at diagnosis is 23 years). More than 65% of patients die from their disease within 2 years of diagnosis. Like MRT, these tumors are characterized by genetic loss of a SWI/SNF complex subunit, SMARCA4. SMARCA4-negative ovarian cancer cells are selectively sensitive to EZH2 inhibition with IC50 values similar to those observed in MRT cells. For example, current treatment of SCCOHT consists of debulking surgery and platinum-based chemotherapeutics and shows a high rate of relapse. Differential diagnosis is broad and includes three ovarian carcinoma subtypes: granulosa cell (sex cord stromal) tumors, dysgerminoma, and high-grade serous tumors.

Standard hematoxylin and eosin (H&E) staining showed SCCOHT to be Rhabdoid-like with sheet-like arrangement of small, tightly packed, monomorphic, highly proliferative, and poorly differentiated cells whereas IHC suggests that SCCOHT is characterized by inactivation of the SMARCA4 gene leading to protein loss, and the non-mutational silencing of SMARCA2 protein. (See, e.g., Karnezis et al., J. Pathol. 2016; 238: 389-400, Jelinic et al. Nat Genet 2014, Witkowski et al., Nat. Genet. 2014; 46: 424-426, Ramos et al. Nat. Genet. 2014; 46: 427-429, Kupryjanczyk et al. Pol. J. Pathol. 2013; 64:238-246, the contents of each of which are incorporated herein by reference in their entireties). Some aspects of this disclosure provide that tumor cells and tumors, e.g., SCCOHT tumors, exhibiting SMARCA4 loss (e.g., as a result of a mutation) and SMARCA2 loss (e.g., as a result of protein loss) are sensitive to HDAC inhibition and can thus effectively be treated with (S)-N-hydroxy-2-(2-(4-methoxyphenyl)butanamido)thiazole-5-carboxamide.

### Epithelioid Sarcoma

Epithelioid sarcoma is a rare soft tissue sarcoma, representing less than 1% of all soft tissue sarcomas. It was first clearly characterized in 1970. The most common genetic mutation found in epithelioid sarcoma is loss of INI-1 (in about 80-90%). Two variants of epithelioid sarcoma have been reported: Distal epithelioid sarcoma is associated with a better prognosis, and affects the upper and lower distal extremities (fingers, hands, forearms, or feet), while proximal epithelioid sarcoma is associated with a worse prognosis, and affects the proximal extremities (upper arm, thigh), and trunk. Epithelioid sarcoma occurs in all age groups but is most common in young adults (median age at diagnosis is 27 years).

Epithelioid sarcoma is associated with a high rate of relapse after initial treatment, and the median survival is less than 2 years when metastatic epithelioid sarcoma is diagnosed. Local recurrences and metastasis occur in about 30-50% of patients, with metastasis typically to lymph nodes, lung, bone, and brain. Treatment of epithelioid sarcoma includes surgical resection as the preferred method of treatment. For inoperable tumors or post-recurrence, conventional chemotherapy and radiation therapy, alone or in combination, are used with relatively low rates of success. About 50% of oncologists consider epithelioid sarcoma to be chemotherapy-insensitive.

The present disclosure relates to a compound (S)-N-hydroxy-2-(2-(4-methoxyphenyl)butanamido)thiazole-5-carboxamide, its pharmaceutically acceptable salt, ester, derivative, analog, prodrug, or solvate thereof.

The disclosure encompasses any physiochemical form (S)-N-hydroxy-2-(2-(4-methoxyphenyl)butanamido)thiazole-5-carboxamide may assume. Non-limiting examples of the physiochemical forms include hydrated forms, solvated forms, crystalline (known or yet to be disclosed), polymorphic crystalline, and amorphous form, etc. Methods of generating such physiochemical forms will be known by one skilled in the art.

The present disclosure also relates to a pharmaceutical composition for treating a histone deacetylase (HDAC)-associated disease. The pharmaceutical composition comprises at least a first active ingredient selected from the group consisting of: (S)-N-hydroxy-2-(2-(4-methoxyphenyl)butanamido)thiazole-5-carboxamide, N-hydroxy-2-(2-(4-methoxyphenyl)butanamido)thiazole-5-carboxamide, and pharmaceutically acceptable salt, ester, derivative, analog, prodrug, or solvate thereof.

In some aspects, (S)-N-hydroxy-2-(2-(4-methoxyphenyl)butanamido)thiazole-5-carboxamide, pharmaceutically acceptable salt, ester, derivative, analog, prodrug, or solvate thereof, is 80-100% of the first active ingredient, by weight, or any percent range in between, e.g., 85-100%, 85-99.99%, 90-99.99%, 90-99.9%, 92.5%-99.9%, 92.5%-99.5%, 95-99.5%, 95-99%, or 97.5-99%, etc. In other aspects, #1a, pharmaceutically acceptable salt, ester, derivative, analog, prodrug, or solvate thereof, is at least 80%, at least 85%, at least 90%, at least 92.5%, at least 95%, at least 97.5%, or at least 99% of the first active ingredient, by weight.

Pharmaceutically acceptable salts include any salt derived from an organic or inorganic acid. Examples of such salts include but are not limited to the following: salts of hydrobromic acid, hydrochloric acid, nitric acid, phosphoric acid and sulphuric acid. Organic acid addition salts include, for example, salts of acetic acid, benzenesulphonic acid, benzoic acid, camphorsulphonic acid, citric acid, 2-(4-chlorophenoxy)-2-methylpropionic acid, 1,2-ethanedisulphonic acid, ethanesulphonic acid, ethylenediaminetetraacetic acid (EDTA), fumaric acid, glucoheptonic acid, gluconic acid, glutamic acid, N-glycolylarsanilic acid, 4-hexylresorcinol, hippuric acid, 2-(4-hydroxybenzoyl) benzoicacid, 1-hydroxy-2-naphthoicacid, 3-hydroxy-2-naphthoic acid, 2-hydroxyethanesulphonic acid, lactobionic acid, n-dodecyl sulphuric acid, maleic acid, malic acid, mandelic acid, methanesulphonic acid, methyl sulpuric acid, mucic acid, 2-naphthalenesulphonic acid, pamoic acid, pantothenic acid, phosphanilic acid ((4-aminophenyl) phosphonic acid), picric acid, salicylic acid, stearic acid, succinic acid, tannic acid, tartaric acid, terephthalic acid, p-toluenesulphonic acid, 10-undecenoic acid or any other such acid now known or yet to be disclosed. It will be appreciated by one skilled in the art that such pharmaceutically acceptable salts may be used in the formulation of a pharmacological composition. Such salts may be prepared by reacting the disclosed compound with a suitable acid in a manner known by those skilled in the art.

In preferred embodiments, the pharmaceutically acceptable salt for #1a is selected from the group consisting of: Na⁺, K⁺, Mg²⁺, Ca²⁺, Zn²⁺ and Al³⁺. In preferred embodiments, the pharmaceutically acceptable salt for #1 is selected from the group consisting of: Na⁺, K⁺, Mg²⁺, Ca²⁺, Zn²⁺ and Al³⁺.

The physical form of the pharmaceutical composition takes depend on a number of factors. For example, the desired method of administration, the physicochemical form taken by the disclosed compound or pharmaceutically acceptable salts thereof. Non-limiting examples of the physical forms include solid, liquid, gas, sol, gel, aerosol, etc. In some embodiments, the pharmaceutical composition consists of the disclosed compound or a pharmaceutically acceptable salt thereof, without any other additive.

In other embodiments, the pharmaceutical composition includes a second active ingredient of a distinct chemical formula from (S)-N-hydroxy-2-(2-(4-methoxyphenyl)butanamido)thiazole-5-carboxamide or N-hydroxy-2-(2-(4-methoxyphenyl)butanamido)thiazole-5-carboxamide. In some aspects, the second active ingredient has the same or a similar molecular target as the target of (S)-N-hydroxy-2-(2-(4-methoxyphenyl)butanamido)thiazole-5-carboxamide or N-hydroxy-2-(2-(4-methoxyphenyl)butanamido)thiazole-5-carboxamide. In other embodiments, the second active ingredient acts upstream of the molecular target of (S)-N-hydroxy-2-(2-(4-methoxyphenyl)butanamido)thiazole-5-carboxamide or N-hydroxy-2-(2-(4-methoxyphenyl)butanamido)thiazole-5-carboxamide with regard to one or more biochemical pathways. In yet other embodiments, the second active ingredient acts downstream of the molecular target of (S)-N-hydroxy-2-(2-(4-methoxyphenyl)butanamido)thiazole-5-carboxamide or N-hydroxy-2-(2-(4-methoxyphenyl)butanamido)thiazole-5-carboxamide with regard to one or more biochemical pathways. Pharmaceutical compositions that include the disclosed compound may be prepared using methodology well known in the pharmaceutical art.

In some embodiments, the pharmaceutical composition includes materials capable of modifying the physical form of a dosage unit. In a nonlimiting example, the composition includes a material that forms a coating that holds in the compound. Non-limiting examples of the materials include sugar, shellac, gelatin, and other inert coating agents.

The present invention is directed to a method of treating a histone deacetylase (HDAC) associated disease in a subject, comprising administering to the subject a composition selected from the group consisting of: (S)-N-hydroxy-2-(2-(4-methoxyphenyl)butanamido)thiazole-5-carboxamide or N-hydroxy-2-(2-(4-methoxyphenyl)butanamido)thiazole-5-carboxamide, and pharmaceutically acceptable salt, ester, derivative, analog, prodrug, or solvate thereof.

Histone acetyltransferases (HAT) impact gene expression by controlling the coiling and uncoiling of DNA around histones. Histone acetyltransferases accomplish this by acetylating lysine residues in core histones leading to less compact and more transcriptionally active chromatin. In contrast, histone deacetylases (HDAC) remove the acetyl groups from lysine residues, leading to a more condensed and transcriptionally silenced chromatin. Reversible modification of the terminal tails of core histones constitutes the major epigenetic mechanism for remodeling of higher-order chromatin structure and controlling gene expression. HDAC inhibitors (HDI) block this action and can result in hyperacetylation of histones, thereby affecting gene expression. Thagalingam S., Cheng K H, Lee H J et al., Ann. N.Y. Acad. Sci. 983: 84-100, 2003; Marks P A. Richon V M, Rifkind R A, J. Natl. Cancer Inst. 92 (15) 1210-16, 2000; Dokmanovic M, Clarke C., Marks P A, Mol. Cancer Res. 5 (10) 981-989, 2007.

Histone deacetylase (HDAC) inhibitors are a class of cytostatic agents that inhibit the proliferation of tumor cells in culture and in vivo by inducing cell cycle arrest, differentiation and/or apoptosis. Acetylation and deacetylation play important roles in the modulation of chromatin topology and the regulation of gene transcription. Histone deacetylase inhibitors induce the accumulation of hyperacetylated nucleosome core histones in many regions of chromatin but affect the expression of only a small subset of genes, leading to transcriptional activation of some genes, but repression of an equal or larger number of other genes. Non-histone proteins such as transcription factors are also the targets for acetylation with varying functional effects. Acetylation enhances the activity of some transcription factors such as the tumor suppressor p53 and the erythroid differentiation factor GATA-1 but may repress the transcriptional activity of others including T cell factor and the co-activator ACTR. Recent studies have shown that the estrogen receptor alpha (ERalpha) can be hyperacetylated in response to histone deacetylase inhibition, suppressing ligand sensitivity and regulating transcriptional activation by histone deacetylase inhibitors. Conservation of the acetylated ERalpha motif in other nuclear receptors suggests that acetylation may play an important regulatory role in diverse nuclear receptor signaling functions. A number of structurally diverse histone deacetylase inhibitors have shown potent antitumor efficacy with little toxicity in vivo in animal models. Several compounds are currently in early phase clinical development as potential treatments for solid and hematological cancers both as monotherapy and in combination with cytotoxics and differentiation agents.

The HDAC enzyme family constitutes a family of 18 genes that can be grouped into four subclasses; classes I-IV, based on their homology to respective yeast orthologs. HDACs, belonging to classes I, II and IV, comprise 11 members, namely HDAC isoforms 1-11, commonly referred to as the classical HDACs, are metal-dependent hydrolases. HDACs of class III, which comprise 7 members, known as sirtuins, namely Sirt 1-7, are NAD+-dependent hydrolases. Class I HDACs are nuclear proteins with ubiquitous tissue expression. Class II and IV HDACs are found in both the nucleus and cytoplasm and exhibit tissue-specific expression. The Class II HDAC family is further subdivided into subclasses IIA and IIB. Class IIA comprises isoforms HDAC4, HDAC5, HDAC7 and HDAC9 while Class IIB comprises isoforms HDAC6 and HDAC10. HDAC6 contains two tandem deacetylase domains and a C-terminal zinc finger domain. HDAC10 is structurally related to HDAC6 but has one additional catalytic domain. Table 1 represents the cellular location and tissue expression of classical HDACs (adapted from Witt, O. et al., Cancer Lett., 277:8-21 (2008)).

TABLE 1
Classical HDACs, Cellular Location and Tissue Expression

| Class | Isoform | Cellular Location | Tissue Expression |
|---|---|---|---|
| Class I | HDAC1 | Nuclear | Ubiquitous |
| | HDAC2 | Nuclear | Ubiquitous |
| | HDAC3 | Nuclear | Ubiquitous |
| | HDAC8 | Nuclear/cytoplasmic | Ubiquitous |
| Class IIA | HDAC4 | Nuclear/cytoplasmic | Heart, smooth |
| | | | muscles, brain |
| | HDAC5 | Nuclear/cytoplasmic | Heart, smooth |
| | | | muscle, brain |
| | HDAC7 | Nuclear/cytoplasmic | Heart, placenta, |
| | | | pancreas, smooth |
| | | | muscle |
| | HDAC9 | Nuclear/cytoplasmic | Smooth muscle, |
| | | | brain |
| Class IIB | HDAC6 | Cytoplasmic | Kidney, liver, heart, |
| | | | pancreas |
| | HDAC10 | Cytoplasmic | Spleen, kidney, liver |
| Class IV | HDAC11 | Nuclear/cytoplasmic | Heart, smooth |
| | | | muscle, kidney, |
| | | | brain |

HDACs play a significant role in both normal and aberrant cell proliferation and differentiation. HDACs have been associated with some disease states involving proliferation, including, but not limited to, cell proliferative diseases and conditions, such as various forms of cancer. (Reviewed in Witt, O. et al., Cancer Lett., 277:8-21 (2008); and Portella A. et al., Nat. Biotechnol., 28:1057-1068 (2010)). Class I and II HDACs have been identified as attractive targets for anticancer therapy. In particular, distinct class I and class II HDAC proteins are overexpressed in some cancers, including ovarian (HDAC1-3), gastric (HDAC2), and lung cancers (HDAC1 and 3), among others. Also, a possible correlation between HDAC8 and acute myeloid leukemia (AML) has been suggested. Concerning class II HDAC proteins, aberrant expression of HDAC6 is induced in some breast cancer cells. Based on their clinical effects, HDAC inhibitors have been identified that suppress tumor cell proliferation, induce cell differentiation, and upregulate crucial genes associated with anti-cancer effects. HDACs have also been implicated in various types of cancers (Bali P, et al., “Inhibition of histone deacetylase 6 acetylates and disrupts the chaperone function of heat shock protein 90: A novel basis for antileukemia activity of histone deacetylase inhibitors,” J. Biol. Chem., 2005 280:26729-26734; Santo L. et al., “Preclinical activity, pharmacodynamic and pharmacokinetic properties of a selective HDAC6 inhibitor, ACY-1215, in combination with bortezomib in multiple myeloma,” Blood, 2012, 119(11): 2579-89), autoimmune or inflammatory diseases (Shuttleworth, S. J., et al., Curr. Drug Targets, 11:1430-1438 (2010)), cognitive and neurodegenerative diseases (Fischer, A., et al., Trends Pharmacol. Sci., 31:605-617 (2010); Chuang, D.-M., et al., Trends Neurosci. 32:591-601 (2009)), fibrotic diseases (Pang, M. et al., J. Pharmacol. Exp. Ther., 335:266-272 (2010)), protozoal diseases (see, e.g., U.S. Pat. No. 5,922,837), and viral diseases (Margolis, D. M. et al., Curr. Opin. HIV AIDS, 6:25-29 (2011)).

In recent years, there has been an effort to develop HDAC inhibitors as cancer treatments and/or as an adjunct therapy. Mark P A. et al. Expert Opinion on Investigational Drugs 14 (12): 1497-1511 (2005). The exact mechanisms by which the compounds may work are unclear, but epigenetic pathways have been studied to help elucidate the exact biological pathways. Claude Monneret, Anticancer Drugs 18(4):363-370 2007. For example, HDAC inhibitors have been shown to induce p21 (WAFI) expression, a regulator of p53′s tumor suppressor activity. Rochon V M. et al., Proc. Natl. Acad. Sci. U.S.A. 97(18): 10014-10019, 2000. HDACs are involved in the pathway by which the retinoblastoma protein (pRb) suppresses cell proliferation. The pRb protein is part of a complex that attracts HDACs to the chromatin so that it will deacetylate histones. Brehm A. et al., Nature 391 (6667): 597-601, 1998. HDAC1 negatively regulates the cardiovascular transcription factor Kruppel-like factor 5 through direct interaction. Matsumura T. et al., J. Biol. Chem. 280 (13): 12123-12129, 2005. Estrogen is well-established as a mitogenic factor implicated in the tumorigenesis and progression of breast cancer via its binding to the estrogen receptor alpha (ERα). Recent data indicate that chromatin inactivation mediated by HDAC and DNA methylation is a critical component of ERα silencing its human breast cancer cells. Zhang Z. et al., Breast Cancer Res. Treat. 94(1): 11-16, 2005.

In some aspects, the composition is administered at 10-400 mg/kg, or any number in between, e.g., 10-350 mg/kg, 20-350 mg/kg, 20-300 mg/kg, 30-300 mg/kg, 30-250 mg/kg, 40-250 mg/kg, 40-200 mg/kg, 50-200 mg/kg, 50-150 mg/kg, 60-150 mg/kg, or 60-100 mg/kg, etc.

In other aspects, the composition is administered about every 4, 8, 12, 16, or 24 hours. In yet other aspects, the composition is administered every 1-24 hours, or any number in between, e.g., 2-24 hours, 2-18 hours, 3-18 hours, 3-16 hours, 4-16 hours, 4-12 hours, 5-12 hours, 5-8 hours, etc.

In some embodiments, the composition further comprises a second active ingredient selected from the group consisting of a chemotherapy drug, an EZH2 inhibitor, a receptor tyrosine kinase inhibitor, CDK4/6 inhibitors, an agent that enhances antigen presentation (“antigen-presentation combination”), an agent that enhances an effector cell response (“effector cell combination”), an agent that decreases tumor immunosuppression (“anti-tumor immunosuppression combination”), and combinations thereof.

Non-limiting examples of the chemotherapy drug include: cis-diamminedichloro platinum (II) (cisplatin), doxorubicin, 5-fluorouracil, taxol, and topoisomerase inhibitors such as etoposide, teniposide, irinotecan and topotecan, etc. Non-limiting examples of EZH2 inhibitor, include tazemetostat (EPZ-6438). Non-limiting examples of receptor tyrosine kinase inhibitors include ponatinib. Non-limiting examples of CDK4/6 inhibitors include Ribociclib, Palbociclib (PD-0332991), Abemaciclib (LY2835219), and Trilaciclib (G1T28).

### Antigen-Presentation Combination

Non-limiting examples of the agent that enhances antigen presentation include: an agent that enhances antigen presentation, an agent that enhances lysis of tumor cells, an agent that stimulates a phagocyte, an agent that disinhibits a phagocyte, an agent that activates a dendritic cell, an agent that activates a macrophage (e.g., a macrophage I), an agent that recruits a dendritic cell, or an agent that recruits a macrophage (e.g., a macrophage I), or a vaccine, etc. In certain non-limiting aspects, the agent that enhances antigen presentation enhances tumor antigen presentation.

Non-limiting examples of the vaccine include: a cell-based vaccine (e.g., a dendritic cell-based vaccine such as Provenge®), or an antigen-based vaccine (e.g., IL-2 in combination with MUC1), etc. A non-limiting example of the agent that enhances lysis of tumor cells is an oncolytic virus. A non-limiting example of the agent that stimulates a phagocyte is a Type I interferon (IFN) activator, for example, a TLR agonist, or a RIG-I-like receptor agonist (RLR), etc. Non-limiting examples of the agent that activates and/or recruits a dendritic cell or a macrophage include: a bi-specific cell engager or a tri-specific cell engager, etc.

In some embodiments, the agent that enhances antigen presentation is selected from the group consisting of: an agonist of Stimulator of Interferon Genes (a STING agonist), an agonist of a Toll-like receptor (TLR), a TIM-3 modulator, a vascular endothelial growth factor receptor (VEGFR) inhibitor, a c-Met inhibitor, a TGF-beta inhibitor, an IDO/TDO inhibitor, an A2AR antagonist, an oncolytic virus, a vaccine, a bi-specific cell engager, a tri-specific cell engager, a bi-specific antibody molecule, a tri-specific antibody molecule, an IDO/TDO inhibitor, and combinations thereof.

Non-limiting examples of TLR include: an agonist of TLR-3, TLR-4, TLR-5, TLR-7, TLR-8, or TLR-9, etc. A non-limiting example of the TIM-3 modulator is an anti-TIM-3 antibody molecule. A non-limiting example of the TGF-beta inhibitor is an anti-TGF-beta antibody. A non-limiting example of the vaccine is a scaffold vaccine. In some aspects, the oncolytic virus expresses a cytokine, for example, GM-CSF, or a CSF (e.g., CSF1, or CSF2), etc. Non-limiting examples of bi- or tri-specific cell engager include: a bi- or tri-specific antibody molecule to CD47 and CD19, with or without an Fc domain.

### Effector Cell Combination

Non-limiting examples of the agent that enhances an effector cell response include: a lymphocyte activator, an agent that activates and/or disinhibits a tumor infiltrating lymphocyte (TIL), an NK cell modulator, an interleukin or an interleukin variant, a bi- or tri-specific cell engager, an NK cell therapy, a vaccine that induces NK cells and an antigen/immune stimulant, an immunomodulator, a T cell modulator, a bispecific T cell engager, an inhibitor of IAP (Inhibitor of Apoptosis Protein), or an inhibitor of target of rapamycin (mTOR), etc.

Non-limiting examples of the lymphocyte activator include: an NK cell activator, or a T cell activator, etc. Non-limiting examples of the tumor infiltrating lymphocyte (TIL) include: an NK cell, or a T cell, etc. A non-limiting example of the NK cell modulator is a modulator (e.g., an antibody molecule) of an NK receptor, for example, a modulator of NKG2A, KIR3DL, NKp46, MICA, CEACAM1, or combinations thereof, etc. Non-limiting examples of the interleukin include: IL-2, IL-15, IL-21, IL-13R, IL-12 cytokine, or a combination thereof, etc. Non-limiting examples of the bi- or tri-specific cell engager include: a bispecific antibody molecule of NKG2A and CD138, or a bispecific antibody molecule of CD3 and TCR, etc. Non-limiting examples of the immunomodulator include: an activator of a costimulatory molecule, or an inhibitor of an immune checkpoint molecule, etc.

In some embodiments, the T cell modulator is a T cell modulator chosen from an inhibitor of a checkpoint inhibitor. Non-limiting examples of the T cell modulator chosen from an inhibitor (e.g., an antibody) of a checkpoint inhibitor include: an inhibitor of PD-1, an inhibitor of PD-L1, an inhibitor of TIM-3, an inhibitor of LAG-3, an inhibitor of VISTA, an inhibitor of diacylglycerol kinases (DKG)-alpha, an inhibitor of B7-H3, an inhibitor of B7-H4, an inhibitor of TIGIT, an inhibitor of CTLA4, an inhibitor of BTLA, an inhibitor of CD160, an inhibitor of TIM1, an inhibitor of IDO, an inhibitor of LAIR1, an inhibitor of IL-12, or a combination thereof, etc.

In other embodiments, the T cell modulator is a T cell modulator chosen from an agonist or an activator of a costimulatory molecule. Non-limiting examples of the T cell modulator chosen from an agonist or an activator of a costimulatory molecule include: an agonistic antibody, an antigen-binding fragment thereof, or a soluble fusion, etc. of GITR, OX40, ICOS, SLAM (e.g., SLAMF7), HVEM, LIGHT, CD2, CD27, CD28, CDS, ICAM-1, LFA-1 (CD11a/CD18), ICOS (CD278), 4-1BB (CD137), CD30, CD40, BAFFR, CD7, NKG2C, NKp80, CD160, B7-H3, or CD83 ligand, etc. A non-limiting example of the bispecific T cell engager is a bispecific antibody molecule that binds to CD3 and a tumor antigen, for example, Epidermal Growth Factor Receptor (EGFR), PSCA, PSMA, EpCAM, or HER2, etc.

### Anti-Tumor Immunosuppression Combination

Non-limiting examples of the agent that decreases tumor immunosuppression include: an agent that modulates the activity and/or level of Treg, macrophage 2, and/or MDSCs, an agent that increases M2 polarization, Treg depletion, and/or T cell recruitment.

Non-limiting examples of the agent that decreases tumor immunosuppression include: an immunomodulator, a CSF-1/1R inhibitor, an IL-17 inhibitor, an IL-1.beta. inhibitor, a CXCR2 inhibitor, an inhibitor of a phosphoinositide 3-kinase, a BAFF-R inhibitor, a MALT-1/BTK inhibitor, a JAK inhibitor, a CRTH2 inhibitor, a VEGFR inhibitor, an IL-15 or a variant thereof, a CTLA-4 inhibitor, an IDO/TDO inhibitor, an A2AR antagonist, a TGF-beta inhibitor, or a PFKFB3 inhibitor, an inhibitor of an immune checkpoint molecule, etc.

Non-limiting examples of the immunomodulator include: an activator of a costimulatory molecule (e.g., a GITR agonist), or an inhibitor of an immune checkpoint molecule (e.g., PD-1, PD-L1, LAG-3, TIM-3, or CTLA-4, etc.), etc. A non-limiting example of the CSF-1/1R inhibitor is an inhibitor of macrophage colony-stimulating factor (M-CSF). A non-limiting example of the inhibitor of a phosphoinositide 3-kinase is PI3K, e.g., PI3K.gamma, or PI3K.delta, etc. Non-limiting examples of the inhibitor of an immune checkpoint molecule include: an inhibitor of PD-1, an inhibitor of PD-L1, an inhibitor of LAG-3, an inhibitor of TIM-3, an inhibitor of CEACAM (e.g., CEACAM-1, CEACAM-3, and/or CEACAM-5, etc.), or an inhibitor of CTLA-4, etc.

In some embodiments, the second active ingredient comprises one or more therapeutic agents that enhance antigen presentation, one or more therapeutic agents that enhance an effector cell response, and/or one or more therapeutic agents that decrease tumor immunosuppression.

In certain embodiments, the second active ingredient is selected from the group consisting of: a STING agonist, a TLR agonist (e.g., a TLR7 agonist), a TIM-3 modulator (e.g., a TIM-3 inhibitor), a GITR modulator (e.g., a GITR agonist), a PD-1 inhibitor (e.g., an anti-PD-1 antibody molecule), a PD-L1 inhibitor, a CSF-1/1R inhibitor (e.g., an M-CSF inhibitor), an IL-17 inhibitor, an IL-1.beta. inhibitor, and combinations thereof.

### Pharmaceutical Formulations

The present disclosure also provides pharmaceutical compositions comprising (S)-N-hydroxy-2-(2-(4-methoxyphenyl)butanamido)thiazole-5-carboxamide described herein in combination with at least one pharmaceutically acceptable excipient or carrier.

A “pharmaceutical composition” is a formulation containing (S)-N-hydroxy-2-(2-(4-methoxyphenyl)butanamido)thiazole-5-carboxamide of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required.

As used herein, the phrase “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the disclosure includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A compound or pharmaceutical composition of the disclosure can be administered to a subject in many of the well-known methods currently used for chemotherapeutic treatment. For example, for treatment of cancers, a compound of the disclosure may be injected directly into tumors, injected into the blood stream or body cavities or taken orally or applied through the skin with patches. The dose chosen should be sufficient to constitute effective treatment but not as high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of (S)-N-hydroxy-2-(2-(4-methoxyphenyl)butanamido)thiazole-5-carboxamide, composition, or pharmaceutical composition thereof effective to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or condition to be treated is cancer, including but not limited to, malignant rhabdoid tumor (MRT), MRT of the ovary (MRTO) and small cell cancer of the ovary of the hypercalcemic type (SCCOHT).

For (S)-N-hydroxy-2-(2-(4-methoxyphenyl)butanamido)thiazole-5-carboxamide of the disclosure, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing (S)-N-hydroxy-2-(2-(4-methoxyphenyl)butanamido)thiazole-5-carboxamide of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds (i.e., (S)-N-hydroxy-2-(2-(4-methoxyphenyl)butanamido)thiazole-5-carboxamide of the disclosure) can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. An effective amount of a pharmaceutical agent is that which provides an objectively identifiable improvement as noted by the clinician or other qualified observer. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

The pharmaceutical compositions can be included in a container, pack, or dispenser together with instructions for administration.

The disclosure further encompasses any physiochemical or sterochemical form that the disclosed compounds may assume. Such forms include diastereomers, racemates, isolated enantiomers, hydrated forms, solvated forms, any known or yet to be disclosed crystalline or amorphous form including all polymorphic crystalline forms. Amorphous forms lack a distinguishable crystal lattice and therefore lack an orderly arrangement of structural units. Many pharmaceutical compounds have amorphous forms. Methods of generating such chemical forms will be well known by one skilled in the art.

The compounds of the present disclosure include possible stereoisomers and include not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-lnterscience, 1994).

Racemates, individual enantiomers, or diasteromers of the disclosed compounds may be prepared by specific synthesis or resolution through any method now known or yet to be disclosed. For example, the compound may be resolved into it enantiomers by the formation of diasteromeric pairs through salt formation using an optically active acid. Enantiomers are fractionally crystallized and the free base regenerated. In another example, enantiomers may be separated by chromatography. Such chromatography may be any appropriate method now known or yet to be disclosed that is appropriate to separate enantiomers such as HPLC on a chiral column.

The compounds of the present disclosure are capable of further forming salts. All of these forms are also contemplated within the scope of the claimed disclosure.

As used herein, “pharmaceutically acceptable salts” refer to derivatives of the compounds of the present disclosure wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

(S)-N-hydroxy-2-(2-(4-methoxyphenyl)butanamido)thiazole-5-carboxamide of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., an acetate, propionate or other ester.

(S)-N-hydroxy-2-(2-(4-methoxyphenyl)butanamido)thiazole-5-carboxamide of the present disclosure can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present disclosure can be delivered in prodrug form. Thus, the present disclosure is intended to cover prodrugs of the presently claimed compounds, methods of delivering the same and compositions containing the same. “Prodrugs” are intended to include any covalently bonded carriers that release an active parent drug of the present disclosure in vivo when such prodrug is administered to a subject. Prodrugs in the present disclosure are prepared by modifying functional groups present in the compound in such a way that the modifications are cleaved, either in routine manipulation or in vivo, to the parent compound. Prodrugs include compounds of the present disclosure wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl) N-Mannich bases, Schiff bases and enaminones of amino functional groups, oximes, acetals, ketals and enol esters of ketone and aldehyde functional groups in compounds of the disclosure, and the like, See Bundegaard, H., Design of Prodrugs, p 1-92, Elesevier, New York-Oxford (1985).

(S)-N-hydroxy-2-(2-(4-methoxyphenyl)butanamido)thiazole-5-carboxamide, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter or arrest the progress of the condition.

The dosage regimen can be daily administration (e.g. every 24 hours) of a compound of the present disclosure. The dosage regimen can be daily administration for consecutive days, for example, at least two, at least three, at least four, at least five, at least six or at least seven consecutive days. Dosing can be more than one time daily, for example, twice, three times or four times daily (per a 24-hour period). The dosing regimen can be a daily administration followed by at least one day, at least two days, at least three days, at least four days, at least five days, or at least six days, without administration.

Techniques for formulation and administration of the disclosed compounds of the disclosure can be found in Remington: The Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Methods of the disclosure for treating cancer including treating a malignant rhabdoid tumor (MRT). In preferred embodiments, methods of the disclosure are used to treat a subject having a malignant rhabdoid tumor of the ovary (MRTO). MRTO may also be referred to as small cell cancer of the ovary of the hypercalcemic type (SCCOHT). In certain embodiments, the MRTO or SCCOHT and/or the subject are characterized as SMARCA4-negative. As used herein SMARCA4-negative cells contain a mutation in the SMARCA4 gene, corresponding SMARCA4 transcript (or cDNA copy thereof), or SMARCA4 protein that prevents transcription of a SMARCA4 gene, translation of a SMARCA4 transcript, and/or decreases/inhibits an activity of a SMARCA4 protein. The SMARCA4-negative status of a cell renders that cell sensitive to EZH2 driven oncogenesis.

Methods of the disclosure may be used to treat a subject who is SMARCA4-negative or who has one or more cells that may be SMARCA4-negative. SMARCA4 expression and/or SMARCA4 function may be evaluated by fluorescent and non-fluorescent immunohistochemistry (IHC) methods, including well known to one of ordinary skill in the art. In a certain embodiment the method comprises: (a) obtaining a biological sample from the subject; (b) contacting the biological sample or a portion thereof with an antibody that specifically binds SMARCA4; and (c) detecting an amount of the antibody that is bound to SMARCA4. Alternatively, or in addition, SMARCA4 expression and/or SMARCA4 function may be evaluated by a method comprising: (a) obtaining a biological sample from the subject; (b) sequencing at least one DNA sequence encoding a SMARCA4 protein from the biological sample or a portion thereof; and (c) determining if the at least one DNA sequence encoding a SMARCA4 protein contains a mutation affecting the expression and/or function of the SMARCA4 protein. SMARCA4 expression or a function of SMARCA4 may be evaluated by detecting an amount of the antibody that is bound to SMARCA4 and by sequencing at least one DNA sequence encoding a SMARCA4 protein, optionally, using the same biological sample from the subject.

All percentages and ratios used herein, unless otherwise indicated, are by weight.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

### EXAMPLES

In order that the invention disclosed herein may be more efficiently understood, examples are provided below. It should be understood that these examples are for illustrative purposes only and are not to be construed as limiting the disclosure in any manner.

### Example 1

### Cell Viability Assay

Cell viability was measured by the CellTiter -GlO® cell viability assay from Promega (Madison, Wis.). The CellTiter-GlO® Luminescent Cell Viability Assay is a homogeneous method to determine the number of viable cells in culture based on quantitation of the ATP present, which signals the presence of metabolically active cells. Following treatment, CellTiter-GlO® is added to treatment wells and incubated at 37° C. luminescence values were measured at using a Molecular Devices Spectramax microplate reader

### Single Agent Studies

Cells were grown to 70% confluency, trypsinized, counted, and seeded in 96 well flat-bottom plates at a final concentration of 2.5×10³-5×10³ cells/well (Day 0). Cells were allowed to incubate in growth media for 24 hours. Treatment with the test agents or standard agents began on Day 1 and continued for 72 hours. At the 72-hour timepoint, treatment containing media was removed. Viable cell numbers are quantified by the CellTiter-GlO® cell viability assay as described above. Results from these studies were used to calculate an IC₅₀ value (concentration of drug that inhibits cell growth by 50 percent of control) for each compound.

### Data Collection

For single agent and combination studies, data from each experiment was collected and expressed as % Cell Growth using the following calculation:

% Cell Growth=(fₜₑₛₜ/f_{vehicle})×100

Where fₜₑₛₜ is the fluorescence of the tested sample, and f_{vehicle} is the fluorescence of the vehicle in which the drug is dissolved. Dose response graphs and IC₅₀ values were generated using Prism 6 software (GraphPad) using the following equation:

Y=(Top−Bottom)/(1+10^{((log IC}50^{-X)-HillSlope)})

Where X is the logarithm of concentration and Y is the response. Y starts at the Bottom and goes to Top with a sigmoid shape.

### Results

SCCOHT is characterized by SMARCA2 and SMARCA4 loss. The three SCCOHT cell lines tested (i.e., BIN67, COV434, and SCCOHT-1) were sensitive to (S)-N-hydroxy-2-(2-(4-methoxyphenyl)butanamido)thiazole-5-carboxamide in cell proliferation assays with IC₅₀ values of 51-293 nM (see Table 2).

TABLE 2

| Cell Line | IC₅₀ (μM) |
|---|---|
| BIN67 | 0.051 |
| COV434 | 0.035 |
| SCCOHT-1 | 0.293 |

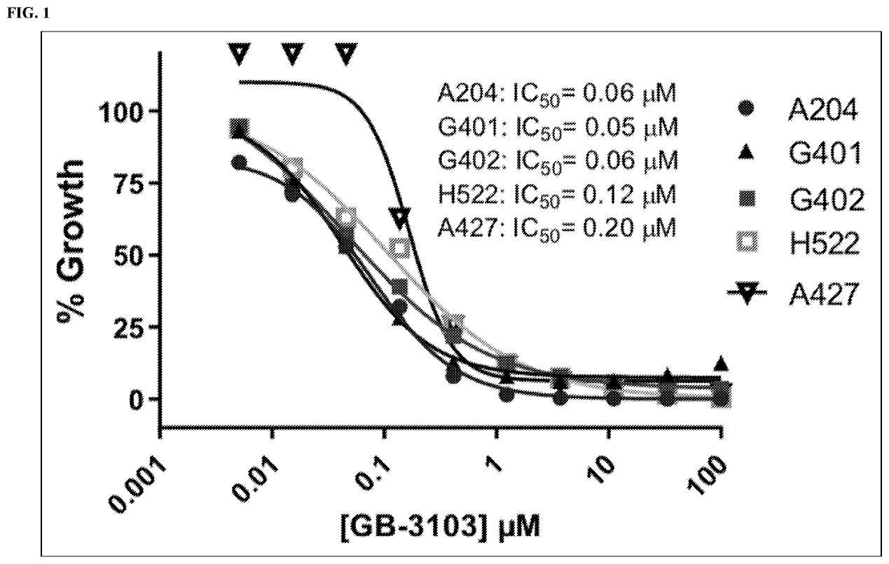

Dual SMARCA2 and SMARCA4 deficient cell lines (i.e., A204, G401, G402, H522, and A427) were also found to be sensitive to (S)-N-hydroxy-2-(2-(4-methoxyphenyl)butanamido)thiazole-5-carboxamide in proliferation assays with IC₅₀ values of 50-200 nM (see FIG. 1).

### Example 2

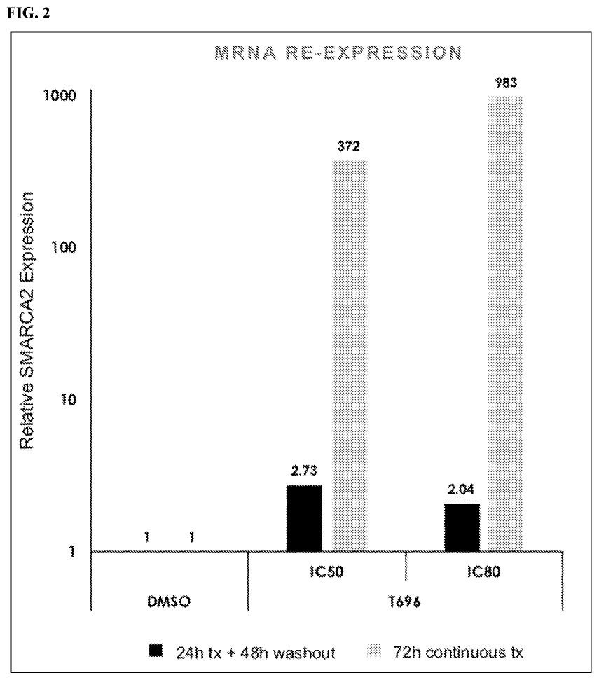

In vitro treatment of BIN-67 cells with (S)-N-hydroxy-2-(2-(4-methoxyphenyl)butanamido)thiazole-5-carboxamide (i.e., GB-3103) demonstrated concentration and time dependent induction of SMARCA2 gene re-expression (see FIG. 2).

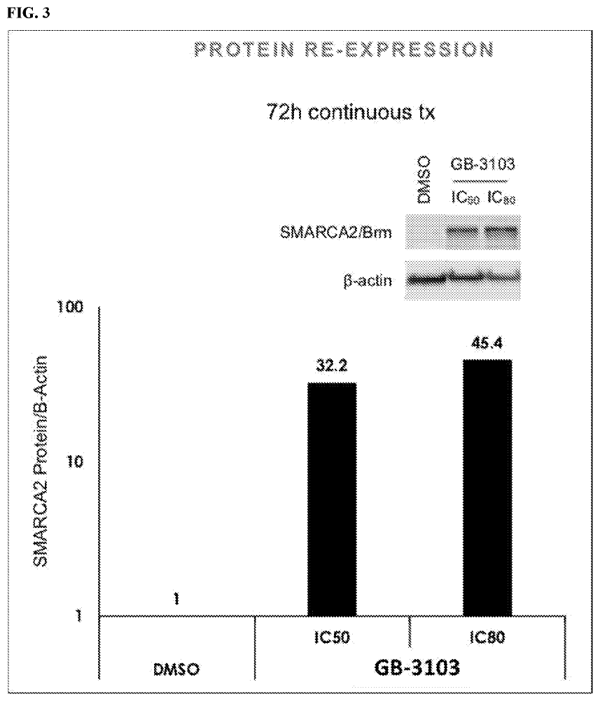

In vitro treatment of BIN-67 cells with (S)-N-hydroxy-2-(2-(4-methoxyphenyl)butanamido)thiazole-5-carboxamide (i.e., GB-3103) also demonstrated concentration and time dependent induction of SMARCA2 protein expression (see FIG. 3).

### Example 3

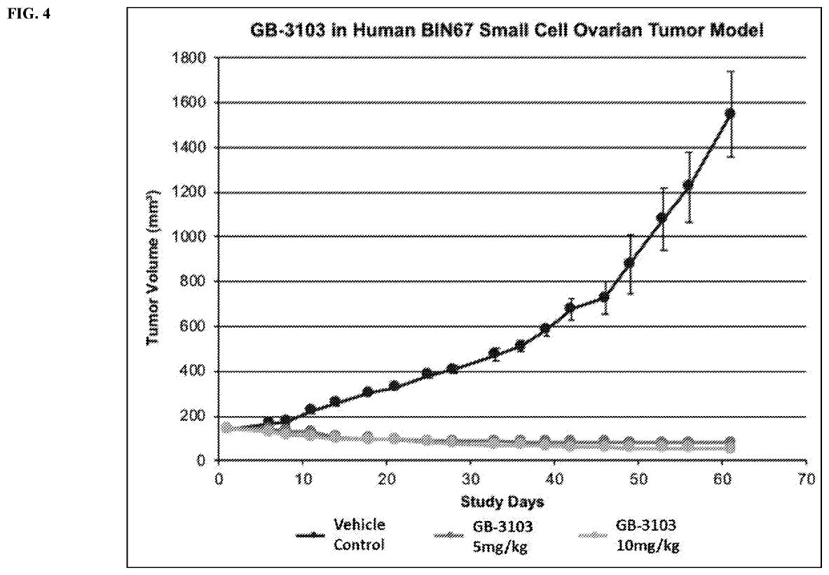

In vivo treatment of tumors in an SCCOHT xenograft model (BIN-67) was evaluated. In vivo xenograft tumors from SCCOHT cell line BIN-67 were dosed with (S)-N-hydroxy-2-(2-(4-methoxyphenyl)butanamido)thiazole-5-carboxamide (i.e., GB-3103) for 60 days. Treated tumors showed statistically significant decreases in volume compared to the vehicle control tumors (see FIG. 4).

### Example 4

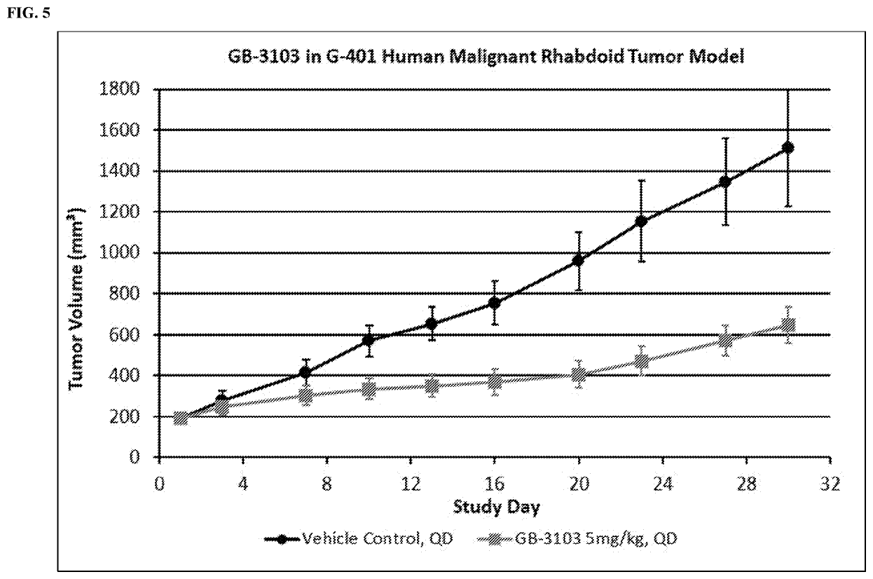

In vivo treatment of tumors in a malignant rhabdoid tumor xenograft model (G401) was evaluated. In vivo xenograft tumors from MRT line G401 were dosed with (S)-N-hydroxy-2-(2-(4-methoxyphenyl)butanamido)thiazole-5-carboxamide (i.e., GB-3103) for 30 days. Treated tumors showed statistically significant decreases in volume compared to vehicle control tumors (see FIG. 5).

### Example 5

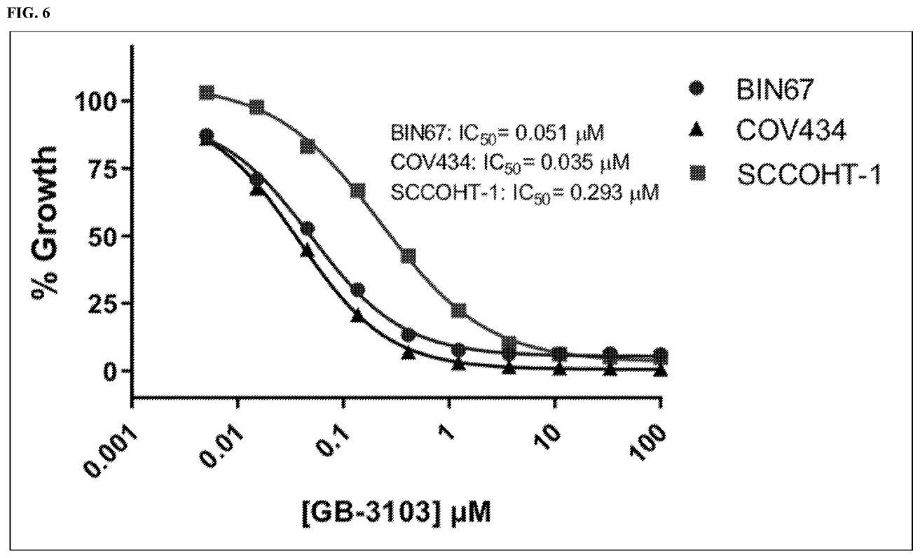

Dual loss of SMARCA4/SMARCA2 ATPases of the SWItch/Sucrose Non-Fermentable (SWI/SNF) complex has been reported in small cell carcinoma of the ovary, hypercalcemic type (SCCOHT) and other tumors. Loss of SMARCA4 is the result of inactivating mutations, and the loss of SMARCA2 results from the absence of mRNA expression. Restoration of either SMARCA4 or SMARCA2 can inhibit the growth of these cancers. The Inventors have evaluated the activity of a novel, structurally rigid, and potent, Class I/IIb HDAC inhibitor, GB-3103, against human SCCOHT and other cells lines deficient in SWI/SNF complex. GB-3103 shows potent anti-proliferative activities with low nM IC₅₀ values against human SCCOHT lines BIN67 (51 nM), COV434 (35 nM) and SCCOHT-1 (293 nM) (see FIG. 6), and SWI/SNF-deficient rhabdoid and lung tumor lines A204 (95 nM), A427 (174 nM), G401 (138 nM), G402 (71 nM), H522 (102 nM) (see FIG. 1).

Treatment of human BIN67 SCCOHT cell line for 72 h with GB-3103 revealed potent concentration- and time-dependent induction of SMARCA2 expression at both mRNA and protein levels (see FIGS. 2 and 3). Treatment of mice bearing G401 human malignant rhabdoid tumor xenografts with GB-3103 at 5 mg/kg, QD resulted in 70% tumor growth inhibition (TGI) compared to vehicle control (P<0.05) (see FIG. 5). Treatment of mice bearing BIN67 human tumor xenografts with GB-3103 at 5 mg/kg and 10 mg/kg, QD resulted in mean tumor regression of 26% and 33%, respectively, at two weeks post-treatment initiation (see FIG. 4).

### Example 6

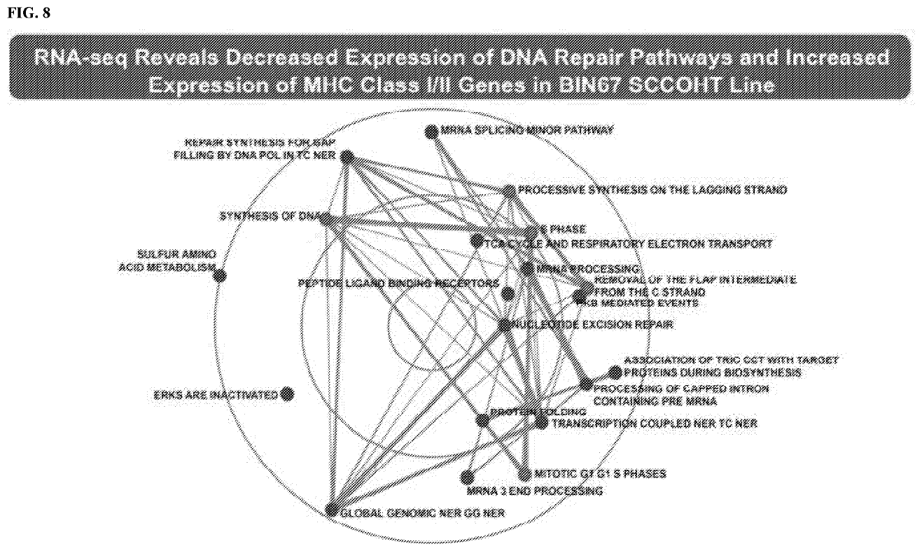

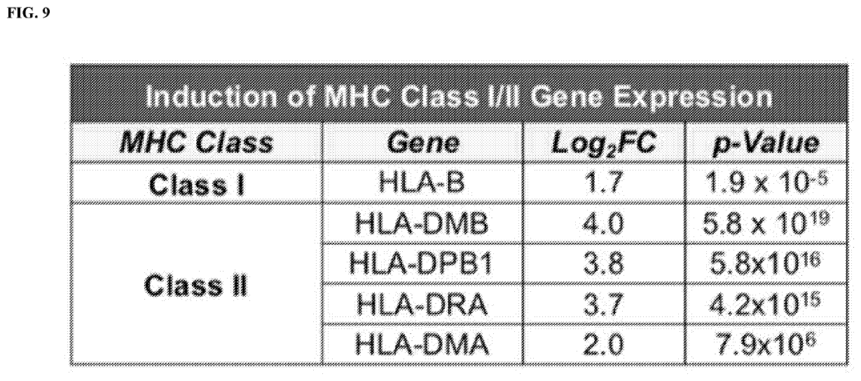

RNA-Seq analyses of BIN67 cells treated with GB-3103 revealed that GB-3103 affects DNA replication and mRNA stability as well as inducing the expression of MHC Class II proteins (see FIGS. 8 and 9).

### Example 7

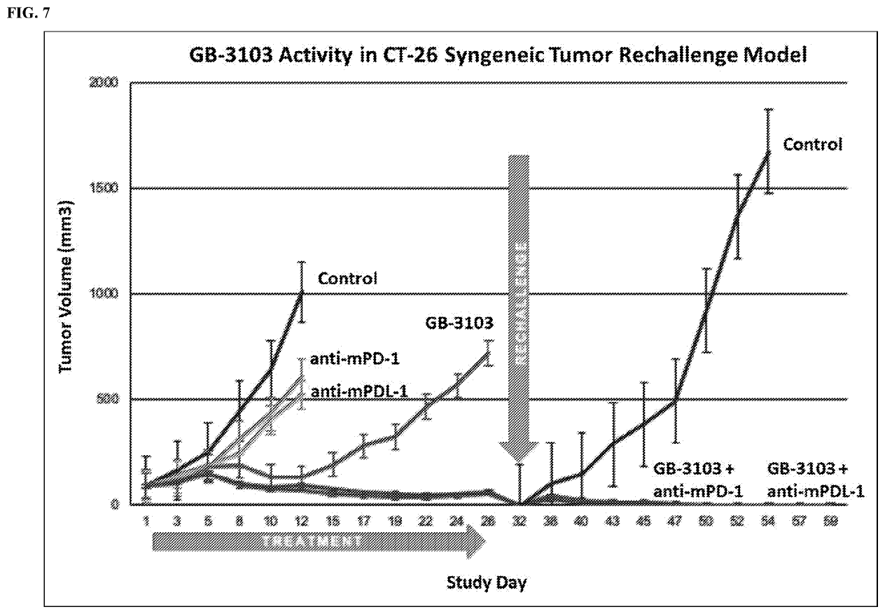

Given the importance of MHC Class II expression and response to checkpoint inhibitor therapies, we tested the activity of GB-3103 alone and in combination with anti-mPD-1 and anti-mPD-L1 antibodies in a syngeneic CT-26 mouse colon cancer model. GB-3103 induced a 93% TGI as a single agent. However, tumor growth resumed on Day 15 and continued to increase until Day 26 (see FIG. 7). Surprisingly, GB-3103 caused regression of established CT-26 tumors when combined with either anti-mPD-1 or anti-mPD-L1 (i.e., antibodies against PD-1 or PD-L1) demonstrating the potent immunomodulatory activity of GB-3103 and the synergistic activity achieved in combination with immune checkpoint inhibitors.

### Example 8

The activity of GB-3103 was determined with HDAC isoforms 1-11 and is shown in Table 3. GB-3103 is the latest generation epigenetic immunomodulator that is a potent HDAC isoform restricted inhibitor demonstrating potent sub-nanomolar inhibition of HDAC3 and an irreversible, sub-nanomolar inhibitor of HDAC6. In contrast to pan-HDAC inhibition, isoform restricted HDAC inhibition revokes immune privilege. HDAC3 has emerged as a key target to enhance immune function:
Decrease in Treg suppressive function
Increase in Natural Killer Cell ligand expression on tumors
Enhance macrophage host defense activity
Upregulation of PD-L1 expression and increased sensitivity to anti-PD1 treatments HDAC6 inhibition has potent immunomodulatory benefits including:
Decrease in expression of the anti-inflammatory cytokine IL-10
Enhanced expression of MHC class I/II genes and increased expression of known tumor antigens
Decreased immunosuppression and enhanced immune function of melanoma patient T-cells

GB-3103 exerts potent effects on DNA repair and induces the expression of class I/II MHC proteins and numerous tumor antigens. GB-3103 demonstrates potent single agent regression of BIN67 SCCOHT tumors and inhibits the growth of G-401 tumors, both harboring SMARCA4/SMARCA2 dual loss ATPases. GB-3103 shows potent activity alone and in combination with anti-PD-1/anti-PD-L1 checkpoint modulators in immune compromised mice and creates a tumor memory response preventing re-growth of tumors. GB-3103 is progressing towards IND-enabling studies for clinical development in patients with genomically defined cancers including those harboring dual loss of SMARCA4/SMARCA2.

GB-3103 is a novel epigenetic immunomodulator with potent anticancer activity against SWI/SNFdeficient cancers. Clinical development of GB-3103 in these genetically defined rare cancers for which no treatments currently exist provides unique clinical and regulatory opportunity for breakthrough therapy designation where approval could be based on smaller single arm clinical studies.

TABLE 3
GB-3103 is a potent inhibitor of Class I/IIb HDACs

| | HDAC1 | HDAC2 | HDAC3 | HDAC4 | HDAC5 | HDAC6 | HDAC7 | HDAC8 | HDAC9 | HDAC10 | HDAC11 |
|---|---|---|---|---|---|---|---|---|---|---|---|
| Compound | IC₅₀ nM | IC₅₀ nM | IC₅₀ nM | IC₅₀ nM | IC₅₀ nM | IC₅₀ nM | IC₅₀ nM | IC₅₀ nM | IC₅₀ nM | IC₅₀ nM | IC₅₀ nM |
| GB-3103 | 1 | 5.3 | 0.5645 | 138 | 73.5 | 0.706 | 34.5 | 133 | 187 | 2.03 | 5780 |

Note:
HDAC1, HDAC2, and HDAC3 are Class I HDACs. HDAC6 and HDAC10 are Class IIb HDACs.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow. Where names of cell lines or genes are used, abbreviations and names conform to the nomenclature of the American Type Culture Collection (ATCC) or the National Center for Biotechnology Information (NCBI), unless otherwise noted or evident from the context.

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1. A method of treating a malignant rhabdoid tumor (MRT), a malignant rhabdoid tumor of the ovary (MRTO), and/or a small cell cancer of the ovary of the hypercalcemic type (SCCOHT) in a subject in need thereof, the method comprising:
administering to the subject a therapeutically effective amount of N-hydroxy-2-(2-(4-methoxyphenyl)butanamido)thiazole-5-carboxamide or an enantiomer, pharmaceutically acceptable salt, solvate, or chemically protected from thereof.

2. The method of claim 1, wherein (S)-N-hydroxy-2-(2-(4-methoxyphenyl)butanamido)thiazole-5-carboxamide or a pharmaceutically acceptable salt thereof is administered to the subject.

3. The method of claim 2, wherein the (S)-N-hydroxy-2-(2-(4-methoxyphenyl)butanamido)thiazole-5-carboxamide or pharmaceutically acceptable salt thereof is administered orally.

4. The method of claim 2, wherein the (S)-N-hydroxy-2-(2-(4-methoxyphenyl)butanamido)thiazole-5-carboxamide or pharmaceutically acceptable salt thereof is administered at a dose of between 1 mg/kg/day and 1600 mg/kg/day.

5. The method of claim 1, wherein the N-hydroxy-2-(2-(4-methoxyphenyl)butanamido)thiazole-5-carboxamide or enantiomer, pharmaceutically acceptable salt, solvate, or chemically protected form thereof is administered at a dose of about 100, 200, 400, 800, or 1600 mg per day.

6. The method of claim 1, wherein the SCCOHT is SMARCA4-negative or the subject is SMARCA4-negative.

7. (canceled)

8. The method of claim 6, wherein SMARCA4 expression is evaluated by a method comprising:
(a) obtaining a biological sample from the subject;
(b) contacting the biological sample or a portion thereof with an antibody that specifically binds SMARCA4; and
(c) detecting an amount of the antibody that is bound to SMARCA4.

9. The method of claim 6, wherein SMARCA4 expression and/or function is evaluated by a method comprising:
(a) obtaining a biological sample from the subject;
(b) sequencing at least one DNA sequence encoding a SMARCA4 protein from the biological sample or a portion thereof; and
(c) determining if the at least one DNA sequence encoding the SMARCA4 protein contains a mutation affecting the expression and/or function of the SMARCA4 protein.

10. The method of claim 1, wherein the subject is less than 40 years of age, less than 30 years of age, less than 20 years of age, or between 20 and 30 years of age, inclusive of the endpoints.

11. The method of claim 1, wherein the N-hydroxy-2-(2-(4-methoxyphenyl)butanamido)thiazole-5-carboxamide or enantiomer, pharmaceutically acceptable salt, solvate, or chemically protected form thereof prevents and/or inhibits proliferation of an SCCOHT cell.

12. A method of treating SCCOHT in a subject in need thereof, the method comprising
administering to the subject in an oral tablet a therapeutically effective amount of N-hydroxy-2-(2-(4-methoxyphenyl)butanamido)thiazole-5-carboxamide or an enantiomer, pharmaceutically acceptable salt, solvate, or chemically protected form thereof.

13. The method of claim 12, wherein (S)-N-hydroxy-2-(2-(4-methoxyphenyl)butanamido)thiazole-5-carboxamide or a pharmaceutically acceptable salt thereof is administered to the subject.

14. The method of claim 13, wherein the (S)-N-hydroxy-2-(2-(4-methoxyphenyl)butanamido)thiazole-5-carboxamide or pharmaceutically acceptable salt thereof is administered at a dose of between 1 mg/kg/day and 1600 mg/kg/day.

15. The method of claim 12, wherein the N-hydroxy-2-(2-(4-methoxyphenyl)butanamido)thiazole-5-carboxamide or enantiomer, pharmaceutically acceptable salt, solvate, or chemically protected form thereof is administered at a dose of about 100, 200, 400, 800, or 1600 mg per day.

16-20. (canceled)

21. A combination comprising:
(S)-N-hydroxy-2-(2-(4-methoxyphenyl)butanamido)thiazole-5-carboxamide or a pharmaceutically acceptable salt thereof; and
an immune checkpoint molecule selected from the group consisting of an inhibitor of PD-1, an inhibitor of PD-L1, an inhibitor of LAG-3, an inhibitor of TIM-3, an inhibitor of CEACAM, and an inhibitor of CTLA-4.

22. The combination of claim 21, wherein the immune checkpoint molecule is an anti-PD-1 antibody molecule.

23. The combination of claim 21, wherein the immune checkpoint molecule is an anti-PD-L1 antibody molecule.

24-28. (canceled)

29. The combination of claim 21, wherein administration of the (S)-N-hydroxy-2-(2-(4-methoxyphenyl)butanamido)thiazole-5-carboxamide or pharmaceutically acceptable salt thereof and the immune checkpoint molecule to a subject in need thereof provides a synergistic effect in the treatment of cancer.

30. The method of claim 1, wherein an MRT is treated.

31. The method of claim 1, wherein an MRTO is treated.
